SEQUENCE LISTING (3 sequences)

SEQ ID NO: 1 - DNA, 19 bp, Artificial Sequence
  Forward primer
caacgcgaagaaccttacc

SEQ ID NO: 2 - DNA, 18 bp, Artificial Sequence
  Reverse primer
acgtcatccccaccttcc

SEQ ID NO: 3 - DNA, 21 bp, Artificial Sequence
  probe labeled at 5′ end with FAM (6-carboxyflourescein) and at 3′ end with BHQ1 (Black Hole QuencherTM dye-1)
acgacaaccatgcaccacctg

(19) United States
(12) Patent Application Publication
(10) Pub. No.: US 2024/0240264 A1
(43) Pub. Date: Jul. 18, 2024

(54) RAPID MILK SAMPLE PREPARATION METHOD COMPATIBLE WITH MOLECULAR TESTS

(71) Applicant: Zoetis Services LLC, Parsippany, NJ (US)

(72) Inventor: Sridhar Velineni, Kalamazoo, MI (US)

(21) Appl. No.: 18/557,791

(22) PCT Filed: Apr. 28, 2022
(86) PCT No.: PCT/US2022/026739

§ 371 Date: Oct. 27, 2023

Related U.S. Application Data
(60) Provisional application No. 63/181,279, filed on Apr. 29, 2021.

(51) Int. Cl. C12Q 1/689; C12Q 1/6806; C12Q 1/6851

(52) U.S. Cl. CPC C12Q 1/689; C12Q 1/6806; C12Q 1/6851; C12Q 2600/16

(57) ABSTRACT

The present invention provides a method of preparing a milk sample for a downstream nucleic acid amplification process. This method includes: subjecting a milk sample comprising at least one suspected bacterial pathogen implicated in mastitis to contact with uncoated magnetic beads; incubating the milk sample in the presence of the uncoated magnetic beads to allow bacterial cells in the milk sample to bind to the beads, thereby forming a bead-bacterium complex; subjecting the bead-bacterium complex to a magnet such that the bead-bacterium complex is separated from a milk sample supernatant; removing the milk sample supernatant; directly resuspending the separated bead-bacterium complex in an aqueous solution to release the bead-bound bacteria into the solution; and employing an aliquot of the bead resuspension or bead-free supernatant following separation of the beads as a template for primer-specific downstream nucleic acid amplification.

### FIELD OF THE INVENTION

The invention relates to molecular tests that allow a dairy farmer to determine pathogens in milk. More particularly, the invention relates to a rapid milk sample preparation method for the point-of-care molecular detection of the etiological pathogens that are associated with mastitis in dairy cows.

### BACKGROUND OF THE INVENTION

Bovine mastitis is the most widespread and costly disease in dairy cattle globally. Mastitis is the inflammation of the mammary gland and udder tissue due to microbial infection or physical trauma. Lack of early diagnosis of bovine mastitis and associated pathogens has adverse economic consequences to the dairy farmer. In the US, mastitis costs the dairy industry about $1.7 to 2.0 billion annually. Although a wide variety of bacterial pathogens have been implicated, the most common pathogens are Staphylococcus aureus, Streptococcus sp., coagulase-negative staphylococci (CNS), Escherichia coli and Mycoplasma sp. Diagnosis of bovine mastitis has been routinely performed utilizing the California Mastitis Test (CMT, a cow-side test), somatic cell count (SCC) and milk cultures (a reference lab test). Typically, if mastitis is diagnosed based on high somatic cell count (SCC), the cow is milked out and treated with an intramammary infusion of antibiotics. Judicious use of antibiotics reduces the likelihood of emerging resistant bacteria and can reduce the duration of treatment a cow may need, which in turn decreases operating costs.

Therefore, accurate bacterial identification is essential to enable appropriate therapeutic intervention as well as planning different management strategies. Except for time-consuming bacterial culture methods, all other available on-farm tests do not detect etiological agents. Routine bacterial culturing at a reference lab usually requires milk samples to be shipped, with a turnaround time of days to weeks. Furthermore, culture tests may indicate a negative result, even from truly sub-clinically infected glands due to the presence of a very low bacterial load when samples are collected. Although molecular methods have proved to be proficient methods of bacterial identification, a rapid and reliable on-farm molecular test to identify the etiological pathogen could provide a significant savings to the dairy farmer. Furthermore, a rapid milk sample preparation method is required to support point-of-care (PoC) molecular mastitis tests, which can be used by untrained dairy personnel. The present invention describes novel and rapid milk sample prep method compatible with downstream real-time PCR for the simultaneous detection of bacterial pathogens in the milk derived from mastitic quarters of cows.

A simple and effective milk sample preparation method is a crucial step in development of PoC molecular test for bovine mastitis. Conventional DNA extraction methods from milk limit the application of molecular diagnostics in on-farm settings. Standard methods of nucleic acid extraction can be broadly categorized into two categories: (a) solid phase extraction (column-based extraction), and (b) nucleic acid capture using magnetic beads. Standard workflows for column-based extraction involve lysis of components of a sample, by way of a lysis buffer, mechanical disruption, heating, or a combination of these techniques. Nucleic acids are released in the lysed sample and are bound to a silica surface in a purification column. Several wash steps clear the silica bound nucleic acids of contaminants and a final elution step releases the purified DNA/RNA from the silica into an aqueous buffer. This method is the most commonly used across molecular labs as many commercial kits and instruments are available for this type of nucleic acid extraction. The extraction process can be performed manually for a handful of samples or can be automated for high-throughput extractions. Magnetic beads-based nucleic acid extraction kits follow a similar principle as column-based extraction (i.e., lysis, capture, washing, and elution), but use magnetic beads to capture the DNA/RNA instead of silica columns. During washing steps, a magnet is used to capture the magnetic beads. After a final elution step, nucleic acids are released back into the solution and the magnetic beads can be discarded. While both of these extraction methods are widely used in research and diagnostics labs, these methods have drawbacks, particularly for use in a PoC setting: 1) expensive instrumentation (e.g. centrifuges) are required for even some manual methods; 2) multiple wash and elution steps; 3) prior training in basic lab techniques are needed to execute the procedure; 4) time consuming; 5) some reagents need refrigeration for storage; and 6) space requirement to house additional equipment, such as a centrifuge and heating blocks. As such, the current drive in molecular diagnostics has been to develop simpler, faster methods of nucleic acid preparation and should involve only a few steps, for example, without the need for any additional equipment and training. Towards this goal, we have developed a simple and easy to use milk sample preparation method which allows detection of pathogens directly from clinical milk samples without any need of nucleic acid purification or additional equipment such as a centrifuge or multiple elution steps.

### SUMMARY OF THE INVENTION

The present invention provides a method of preparing a milk sample for a downstream nucleic acid amplification process. This method includes: a) subjecting a milk sample comprising at least one suspected bacterial pathogen implicated in mastitis to contact with uncoated magnetic beads; b) incubating the milk sample in the presence of the uncoated magnetic beads to allow bacterial cells in the milk sample to bind to the beads, thereby forming a bead-bacterium complex; c) subjecting the bead-bacterium complex to a magnet such that the bead-bacterium complex is separated from a milk sample supernatant; d) removing the milk sample supernatant; e) directly resuspending the separated bead-bacterium complex in an aqueous solution to release the bead-bound bacteria into the solution; and f) employing an aliquot of the bead resuspension from e) as a template for primer-specific downstream nucleic acid amplification. In one embodiment, the method further includes subjecting the bead resuspension from f) to a magnet to separate the beads from a bead-free supernatant, wherein an aliquot of the bead-free supernatant is used as the template for the primer-specific downstream nucleic acid amplification.

In one embodiment, the milk sample is a raw milk sample. In another embodiment, the raw milk sample is from a mastitic quarter of a dairy cow.

In a further embodiment, the separated bead-bacterium complex in step e) of the method is directly resuspended in a buffered aqueous solution that provides a suitable chemical environment for activity of DNA polymerase. In one embodiment, the pH of the buffered aqueous solution is between 8.0 and 9.5. In a further embodiment, the buffered aqueous solution is stabilized by Tris-HCl, Tris-H₃PO₄, MOPS (3-(N-morpholino)propanesulfonic acid), HEPES (4-(2-hydroxyethyl)-1-piperazineethanesulfonic acid), or combinations thereof.

In one embodiment, the buffered aqueous solution used to resuspend the separated bead-bacterium complex is stabilized by Tris-HCl or Tris-H₃PO₄ which is present at about 10 to about 50 mM and at a pH of about 8.0 to about 9.0. In a specific embodiment, the buffered aqueous solution is 25 mM Tris-HCl, pH 8.5.

In some embodiments, the buffered aqueous solution further includes a chelating agent, a salt, a detergent, or combinations thereof. In one embodiment, the chelating agent is ethylenediaminetetraacetic Acid (EDTA), ethylene bis(oxyethylenenitrilo)tetraacetic acid (EGTA), or sodium 4-aminosalicylate. In a further embodiment, the detergent is tricosaethylene glycol dodecyl ether (Brij L23), octoxynol-1 (Triton X-100), polyoxyethylene sorbitan monolaurate (Tween-20), triethanolamine (TEA), 3-((3-cholamidopropyl) dimethylammonio)-1-propanesulfonate (CHAPS), Sodium Dodecyl Sulfate (SDS) or combinations thereof. In another embodiment, the salt is selected from sodium chloride (NaCl), magnesium chloride (MgCl₂), potassium chloride (KCl), or combinations thereof.

In one embodiment of the method, the milk sample and the uncoated magnetic beads are mixed prior to the incubation step. In another embodiment, the incubation is for about 5 to about 10 minutes at room temperature.

In a further embodiment, the steps of preparing the milk sample are carried out in a tube. In one embodiment, the tube is placed in a magnetic stand to capture the magnetic beads.

In one embodiment, the uncoated magnetic beads comprise silica-like surface chemistry. In another embodiment, the uncoated magnetic beads are surface derivatized with lectin or with free amine groups or with streptavidin.

In one embodiment, the template of step f) is analyzed by real-time PCR using primers specific to a bacterial pathogen implicated in mastitis. In one embodiment, the bacterial pathogen implicated in mastitis is selected from Staphylococcus aureus, Streptococcus sp., or coagulase-negative staphylococci (CNS). In a further embodiment, the aliquot of the bead resuspension or the aliquot of the bead-free supernatant is transferred to a PCR tube or microfluidic PCR cartridge.

The present invention also provides a point-of-care (PoC) molecular test that allows identification of an etiological pathogen in a milk sample, the molecular test comprising the use of a template for primer-specific nucleic acid amplification that has been prepared according to any one of the above described embodiments of the milk sample preparation method. In one embodiment, the milk sample is from a mastitic quarter of a dairy cow. In another embodiment, the molecular test is real-time PCR.

### BRIEF DESCRIPTION OF THE DRAWINGS

FIG. 1 is a schematic representation of workflow embodiments for a rapid milk sample preparation according to the present invention.

### DETAILED DESCRIPTION OF THE INVENTION

### Definitions

Throughout this specification, unless the context requires otherwise, the words “comprise,” “comprises,” and “comprising” will be understood to imply the inclusion of a stated step or element or group of steps or elements but not the exclusion of any other step or element or group of steps or elements.

The term “uncoated magnetic beads” as used throughout the specification means magnet beads or magnetic particles that are not coated with an immunoglobulin or peptide specific for the target bacterial pathogen implicated in mastitis, such as Staphylococcus aureus, Streptococcus sp. or coagulase-negative staphylococci (CNS). It is to be understood, however, that the magnetic beads or particles can be surface derivatized with other components that will allow the beads to bind to the bacterial cells and form a bead-bacterium complex. For example, in one embodiment, the surface of the beads can be derivatized with silica-like chemistry (silanol groups). Without wishing to be bound by any one theory, it is believed that magnetic beads with such silica-like chemistry binds to the bacteria in the milk sample via weak Vander Waals forces. In another embodiment, the magnetic beads or particles are lectin-derivatized. The lecithin binds to carbohydrates present on the surface of the bacterial cells in the milk sample. Alternatively, streptavidin can be immobilized to the surface of the magnetic beads. The streptavidin binds to extracellular biotin on the surface of the bacterial cells in the milk sample. The streptavidin-biotin binding is a protein-ligand interaction. Alternatively, the surface of the magnetic beads can be functionalized with free amine groups, which are ready to couple with a ligand. For example, the amine groups interact with proteins localized on the surface of the bacterial cells in the milk sample.

The term “directly resuspending the separated bead-bacterium complex” as used in the specification means that following removal of the milk sample supernatant, the magnetic beads with the bound bacteria are subject to being resuspended in the aqueous buffer in the absence of any washing of the bead-bacterium complex beforehand.

The terms “nucleic acid”, “polynucleotide”, “nucleic acid molecule” and the like may be used interchangeably herein and refer to a series of nucleotide bases (also called “nucleotides”) in DNA and RNA. The nucleic acid may contain deoxyribonucleotides, ribonucleotides, and/or their analogs. The term “nucleic acid” includes, for example, single-stranded and double-stranded molecules. A nucleic acid can be, for example, a gene or gene fragment, exons, introns, a DNA molecule (e.g., genomic DNA), an RNA molecule (e.g., mRNA), recombinant nucleic acids, plasmids, and other vectors, primers and probes. Both 5′ to 3′ (sense) and 3′ to 5′ (antisense) polynucleotides are included.

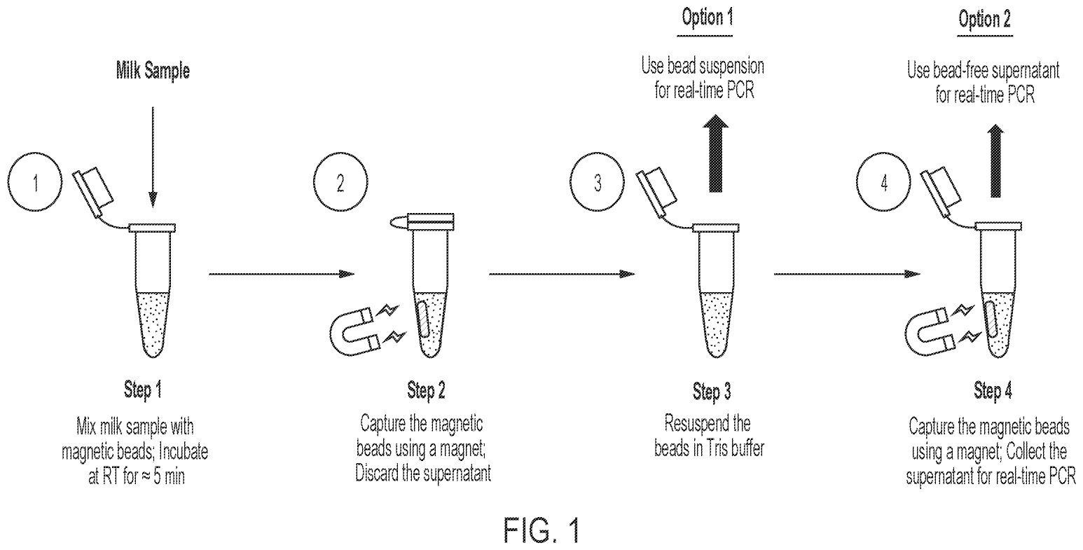

As described above, the invention provides a method of preparing a milk sample for a downstream nucleic acid amplification process. Referring now to FIG. 1, a milk sample is subjected to contact with uncoated magnetic beads. The milk sample is suspected of including at least one suspected bacterial pathogen implicated in mastitis, such as Staphylococcus aureus, Streptococcus sp., or coagulase-negative staphylococci (CNS). The milk sample is incubated in the presence of the uncoated magnetic beads in a tube for a sufficient time period to allow the bacterial cells in the milk sample to bind to the beads, thereby forming a bead-bacterium complex. The bead-bacterium complex which has been thus formed is then subjected to a magnetic force, such as by placement on a magnetic rack. This allows the beads with the bound bacterial cells to be pelleted and separated from a milk sample supernatant, which is then removed from the tube. The separated bead-bacterium complex in the tube is then directly resuspended in the absence of any washing steps beforehand in an aqueous solution to release the bead-bound bacteria into the solution. Next, one of two options can be taken. Option 1 is that an aliquot of the bead resuspension can be directly employed as a template for primer-specific downstream nucleic acid amplification (e.g., real-time PCR) to detect the presence of the bacterial pathogen. Option 2 is that the bead resuspension is subjected to a magnet in order to separate the beads from a bead-free supernatant, wherein an aliquot of the bead-free supernatant is used as the template for the primer-specific nucleic acid amplification (e.g., real-time PCR). It is to be understood that a “bead-free supernatant” might still contain residual amounts of beads, i.e., it is substantially free of beads or includes no beads. In one embodiment, less than 0.1% v/v of residual beads could be present in a “bead-free supernatant”.

The present invention provides a point-of-care (PoC) molecular test that allows identification of an etiological pathogen in a milk sample. This molecular test employs a template for primer-specific nucleic acid amplification that has been prepared according to the milk sample preparation method described herein.

As illustrated in FIG. 1, the user at the point-of-care setting (e.g., a dairy farmer, veterinarian, or veterinary technician) carries out the milk sample preparation method in only 3 to 4 steps. Also, no special instrumentation is required to carry out the method, other than a magnet or magnetic separation rack. In addition, the method does not require a nucleic acid purification step. Furthermore, the sample preparation method can be carried out in a single tube, such as a microfuge tube. In contrast, in commercially available magnetic bead-based kits, after a lysis step, magnetic particles are used to bind nucleic acid (DNA/RNA) followed by washing steps to remove any unbound impurities. In such kits, a final elution step involves addition of buffer to elute nucleic acid bound to magnetic bead. This workflow is labor intensive, involving multiple wash steps with centrifugation between each wash which requires a high level of technical expertise to minimize loss of magnetic beads during multiple washing steps, thus making it difficult to use these kits at point-of-care.

A point-of-care (PoC) molecular test that allows the dairy farmer or veterinarian to quickly determine the etiological pathogen in milk collected from mastitic quarters of a cow is needed. The milk sample preparation method of this invention supports such a PoC molecular mastitis test by allowing the direct detection of pathogens from clinical milk samples without the need for a nucleic acid purification step. In one embodiment, this method involves mixing of magnetic beads with clinical milk samples to trap bacterial cells and subsequent incubation of the mixture at room temperature for ≈5 minutes. In one embodiment, following incubation, the magnetic beads are pelleted, and the milk sample is aspirated using a magnetic separation rack. In another embodiment, the magnetic bead pellet is resuspended in Tris buffer (pH 8.5) and an aliquot of the bead suspension or bead-free supernatant is used directly as template for amplification by downstream real-time PCR.

In one embodiment, the magnetic beads employed in the methods of this invention include oxides of any suitable magnetic material or combination of materials, such as magnetite, ulvospinel, hematite, ilmenete, maghemite, jacobsite, trevorite, magnesioferrite, pyrrhotite, greigite, troilite, goethite, lepidocrocite, feroxyhyte, iron, nickel, cobalt, awaruite, and wairauite.

In one desired embodiment, the magnetic beads include a divalent oxide, a trivalent oxide, or a combination thereof. In one preferred embodiment, the magnetic beads include gamma Fe₂O₃ and Fe₃O₄ throughout the bead.

The magnetic material may be further combined with a polymer to form the beads. For example, magnetic polymer beads are composed of magnetic nano- or microparticles embedded in a polymer matrix. The size of beads can vary from one hundred nanometers to a few millimeters. Synthesis of magnetic polymer beads can be performed by three general ways. In the first one the magnetic particles are synthesized inside polymer matrix. In the second one polymer is synthesized in the presence of magnetic particles. In the third one the beads are prepared from pre-formed polymer and magnetic particles. The beads can have different structures. In one kind of beads the magnetic particles are homogeneously distributed in the volume of polymer matrix. Other kinds of beads are characterized by a core-shell structure (polymer core-magnetic shell or magnetic core-polymer shell. Also, mixed systems can be prepared, where the core-shell particles are homogeneously dispersed in polymer matrix.

As described above, the magnetic beads employed in the milk sample preparation method according to this invention are uncoated magnetic beads or uncoated magnetic particles. This means that their surface is not coated with an antibody or peptide specific for the target bacterial pathogen implicated in mastitis, such as Staphylococcus aureus, Streptococcus sp. or coagulase-negative staphylococci (CNS). It is to be understood, however, that the magnetic beads or particles can be surface derivatized with other components that will allow the beads to bind to the bacterial cells and form a bead-bacterium complex.

In one embodiment, the magnetic beads are uniform, monosized ferrimagnetic beads, about 1 μm in diameter which are composed of cross-linked polystyrene combined with evenly distributed magnetic material and silica-like surface chemistry (silanol groups). Such beads are sold commercially. For example, the example section describes the use of Dynabeads® MyOne™ Silane (Thermo Fisher Scientific, Waltham, MA). However, the present invention is not limited to this embodiment.

In another embodiment, the magnetic beads can comprise streptavidin on their surface. Such beads are sold commercially. For example, Dynabeads® MyOne Streptavidin T1 beads (Thermo Fisher Scientific, Waltham, MA) are superparamagnetic beads, 1 μm in diameter, with a monolayer of covalently coupled recombinant streptavidin and a hydrophobic surface. Also, Dynabeads M-280 Streptavidin beads (Thermo Fisher Scientific, Waltham, MA) are 2.8 μm magnetic beads with covalently coupled recombinant streptavidin and a hydrophobic surface. However, the present invention is not limited to these.

In yet another embodiment, the magnetic beads are lectin-derivatized. Such beads are commercially available. For example, the present inventors used the following two types of lectin-derivatized beads from GlycoMatrix (Dublin, Ohio): 1) Concanavalin A (Jackbean) Lectin (Con A)-MagneZoom™ beads and 2) Triticum vulgaris (Wheat) Lectin (WGA)-MagneZoom™ beads, although the present invention is not limited to these.

In a still further embodiment, the magnetic beads are derivatized with free amine groups. Such beads are commercially available. For example, MagnaBind™ Amine Derivatized Beads are available from Thermo Fisher Scientific, Waltham, MA, although the present invention is not limited to these.

In one embodiment, the milk sample is employed at a concentration of about 95% v/v to about 98.75% v/v. The magnetic beads are employed at a concentration of about 1.25% v/v to about 5.0% (v/v). The term “(v/v)” shall mean volume per volume according to its usual meaning.

The milk sample can be a raw milk sample from a mastitic quarter of a dairy cow. In one embodiment, the uncoated magnetic beads are mixed with the milk sample and then incubated at room temperature for about 5 minutes to allow the beads to bind the bacterial cells in the milk sample, thus forming the bead-bacterium complex. In one embodiment, the milk sample is present at about 95% v/v and the magnetic beads are present at about 5% v/v during the mixing and incubation.

After the incubation of the milk sample with the magnetic beads, the beads are brought into contact with a magnet. In one embodiment, this magnet is in the form of a magnetic stand. Magnetic stands are available commercially. For example, the Ambion® Single Tube Magnetic Stand (Thermo Fisher Scientific, Waltham, MA) accommodates one 1.5 mL microfuge tube and the Invitrogen™ MagnaRack™ Magnetic Separation Rack accommodates 24×1.5 mL microcentrifuge tubes. With these stands/racks, as with any magnetic stand/rack, centrifugation is replaced by the attraction between capture beads in a solution and the magnet in the stand, resulting in the beads collecting on one side of the microfuge tube. Thus, the magnetically-captured beads including the bound bacterial cells from the milk sample can be quickly and efficiently separated from a milk sample supernatant containing the milk sample's other components. The milk sample supernatant is removed from the tube, such as by decanting or aspiration which can be done manually using a clean disposable pipette or automated lab pipette with a clean tip, such as a 200 μL tip.

The magnetically separated beads including the bound bacterial cells may be alternatively referred to herein as the bead-bacterium complex. After the milk sample supernatant has been removed from the tube, the bead-bacterium complex is next resuspended in an aqueous solution, which the inventors envision could simply be distilled water. However, in one embodiment, the bead-bacterium complex is resuspended in a buffered aqueous solution that provides a suitable chemical environment for activity of DNA polymerase.

In one embodiment, the pH of buffered aqueous solution used to resuspend the bead-bacterium complex is between 8.0 and 9.5. In another embodiment, the buffered aqueous solution is stabilized by Tris-HCl, Tris-H₃PO₄, MOPS (3-(N-morpholino)propanesulfonic acid), HEPES (4-(2-hydroxyethyl)-1-piperazineethanesulfonic acid), or combinations thereof. In a further embodiment, the buffered aqueous solution is stabilized by Tris-HCl or Tris-H₃PO₄ which is present at about 10 to about 50 mM and at a pH of about 8.0 to about 9.0. In a still further embodiment, the buffered aqueous solution is 25 mM Tris-HCl, pH 8.5.

In still further embodiments, the buffered aqueous solution used to resuspend the bead-bacterium complex further comprises a chelating agent, a salt, a detergent, or combinations thereof. Such components are typically included in PCR buffers. In one embodiment, the chelating agent is ethylenediaminetetraacetic Acid (EDTA), ethylene bis(oxyethylenenitrilo)tetraacetic acid (EGTA), or sodium 4-aminosalicylate. In another embodiment, the detergent is tricosaethylene glycol dodecyl ether (Brij L23), octoxynol-1 (Triton X-100), polyoxyethylene sorbitan monolaurate (Tween-20), triethanolamine (TEA), 3-((3-cholamidopropyl) dimethylammonio)-1-propanesulfonate (CHAPS), Sodium Dodecyl Sulfate (SDS) or combinations thereof. In yet another embodiment, the salt is selected from sodium chloride (NaCl), magnesium chloride (MgCl₂), potassium chloride (KCl), or combinations thereof.

As described herein and as illustrated in FIG. 1, an aliquot of the bead resuspension can be employed as a template for primer-specific downstream nucleic acid amplification. Alternatively, as also illustrated in FIG. 1, the bead resuspension can be subjected to a magnet in order to separate the beads from a bead-free supernatant, wherein an aliquot of the bead-free supernatant is used as the template for the primer-specific downstream nucleic acid amplification. Although acceptable results were obtained with each of these two options, the present inventors found that the performance of the bead-free supernatant was superior when compared to the bead suspension as the template (Example 6, Table 2)

The template obtained using the sample preparation process of this invention may be amplified with the polymerase chain reaction (PCR) which specifically amplifies target sequences to detectable amounts. In one desired embodiment, a suitable nucleic acid detection method for use in conjunction with the present invention is a real-time quantitative PCR method, which can be dye-based or probe-based. Probe-based quantitative PCR (qPCR) uses real-time fluorescence from 5′-3′ exonuclease cleavage of a fluorescently-labeled, target-specific probe to measure DNA amplification at each cycle of a PCR. Because probe-based qPCR is typically more specific than dye-based qPCR, it is often the foundational technology employed in qPCR diagnostic assays. Probe designs vary but the most common type, hydrolysis (e.g., TaqMan®) probes, incorporate a 5′ reporter fluorophore and a 3′ quencher on a short oligonucleotide complementary to the target sequence. Fluorescence resonance energy transfer (FRET) prohibits emission of the fluorophore while the oligo probe is intact. During each PCR cycle, the 5′ flap endonuclease domain of Taq DNA polymerase hydrolyzes the probe as the primer is extended and the target sequence is amplified. This cleavage event separates the reporter fluorophore from the quencher and results in an amplification-dependent increase in fluorescence. Probe-based qPCR allows multiple targets to be quantified in a single reaction (multiplexing) by using a unique fluorescent dye for each amplicon-specific probe. Software exists for designing PCR primers. For example, PCR primers can be designed with Primer Express© software (Applied Biosystems) or PrimerQuest™ Tool (Integrated DNA Technologies).

The following examples and the tables therein are provided to aid the understanding of the present invention, the true scope of which is set forth in the appended claims. It is understood that modifications can be made in the procedures set forth without departing from the spirit of the invention.

### EXAMPLES

### Example 1—Milk Sample Collection

Milk samples were aseptically collected from a mastitic quarter of dairy cows (n=68) at the milk parlors of different dairies. Sampling methods followed standard recommendations by the National Mastitis Council's Laboratory Handbook on Bovine Mastitis. Briefly, the first streams of milk from each quarter were discarded for mammary gland stimulation, and subsequently the teats were dipped in iodine tincture. Then teats were cleaned and disinfected using 70% ethanol, the first three streams were discarded, and the milk samples were collected into sterile plastic tubes without preservative. Samples were kept on ice until transported to the laboratory, an aliquot was separated for culture and MALDI-TOF analysis, and the remaining sample was stored at −80° C. for real-time PCR analysis using the milk sample preparation method according to the present invention.

### Example 2—Microbiological Culture

To identify suitable samples for downstream molecular analysis, an agar-plate culture system was used for the detection in each milk sample of the target bacterial pathogens that are commonly associated with mastitis. An aliquot of milk samples (100 μL) was plated on the surface of blood agar plates and were aerobically incubated at 37° C. overnight and subsequently read to estimate the bacterial load (CFU/mL). Subsequently, the bacterial population from all study samples were further typed at the genus- and species-level by MALDI-TOF analysis.

### Example 3—Rapid Milk Sample Preparation Method

In one embodiment, a milk sample preparation method according to the present invention involved mixing of 10 μL of magnetic beads with silica-like surface chemistry (Dynabeads™ MyOne™ Silane; Thermo Fisher Scientific, Waltham, MA) with 200 μL of clinical milk sample, followed by incubation at room temperature for ≈5 minutes. After incubation, a magnetic separation rack was used to capture the magnetic bead pellet and the milk supernatant was aspirated. Then, the magnetic bead pellet was resuspended in 100 μL of 25 mM Tris buffer (pH 8.5). An aliquot (10 μL) of the bead suspension or otherwise bead-free supernatant which was separated from the beads by use of the magnetic rack again was used directly as the template for amplification by real time PCR. FIG. 1 depicts embodiments of a workflow of this novel method.

### Example 4—Pathogen Specific Real-Time PCR Assays

An earlier study validated the following primers and probe as specific to Gram-positive bacteria: Forward primer (FP): 5′ CAACGCGAAGAACCTTAC C 3′ (SEQ ID NO: 1); Reverse primer (RP): 5′ ACGTCATCCCCACCTTCC 3′ (SEQ ID NO: 2); and probe: 5′ FAM-ACGACAACCATGCACCACCTG BHQ1 3′ (SEQ ID NO: 3) (Wu et al., 2008 Journal of Clinical Microbiology. 46(8):2613-2619) and were selected to evaluate a rapid milk sample preparation method according to the present invention. These Gram-positive specific primers and probes, which were employed in the downstream real-time PCR, involved the 16S rRNA gene that allows simultaneous detection and discrimination of clinically relevant Gram-positive and Gram-negative bacteria. Gram-positive bacterial pathogens implicated in mastitis include Staphylococcus aureus and coagulase-negative staphylococci (CNS), and should be detected in milk samples taken from mastitic quarters of dairy cows in the downstream real-time PCR.

Prior to assay development, both primers and the probe were synthesized (Integrated DNA Technologies, IA) and a 20× stock solution was prepared by mixing 20 μL of the forward primer (FP), 20 μL of the reverse primer (RP), and 10 μL of the probe with 150 μL of IDT DNA buffer (Integrated DNA Technologies, Inc., Coralville, Iowa).

The real-time PCR assays were carried out in a reaction mixture containing 1× TaqPath ProAmp Master Mix (Applied Biosystems, CA) with 1.5× or 1.0× primer-probe mix for 10 μL of target magnetic bead suspension or magnetic bead-free supernatant prepared by the rapid milk sample preparation method as template, respectively. DNase-RNase free water to make up the final volume to 25 μL. Reaction mixtures were thermally cycled once at 60° C. for 30 sec, 95° C. for 10 min, followed by 45 times at 95 ºC for 15 sec; 60 ºC for 60 sec. The amplicons were subsequently detected in real-time using a CFX96 qPCR machine (Bio-Rad Laboratories, Hercules, CA).

Diagnostic sensitivity and specificity along with Jeffrey's 95% CI were also estimated by testing clinical milk samples positive for Gram-positive bacterial pathogens (S. aureus and coagulase-negative staphylococci) that are associated with bovine mastitis (Table 1 below). Rapid milk sample preparation followed by real-time PCR testing was performed as described above and the amplicons were then detected in real-time by CFX96 qPCR machine (Bio-Rad Laboratories, Hercules, CA) instrument.

### Example 5—Optimization and Performance of Milk Sample Preparation Method

The method of this invention involves the use of magnetic beads to bind to the bacteria in the milk sample, possibly also binding free DNA and other host cells present in the milk samples. Different types of magnetic beads were evaluated, which included simple magnetic beads (Dynabead™ MyOne™ Silane)), or other surface derivatized beads (lectin-magnetic, streptavidin and amine derivatized magnetic beads) and immuno-magnetic beads (magnetic beads coupled with antibodies). Among the different magnetic beads tested, Dynabeads™ with silica-like surface chemistry, Dynabeads™ with streptavidin, lectin-derivatized, and amine-derivatized magnetic beads appeared promising. Lectin-derivatized magnetic beads may have specificity to sugars located on bacterial surfaces, such as N-acetyl glucosamines. The Dynabeads™ with streptavidin bind to extracellular biotin on the surface of the bacterial cells. Also, without wishing to be bound by any one theory, the hypothesis driving the inclusion of simple magnetic beads (Dynabeads™ with silica-like surface chemistry) is that their binding with bacterial cells may be random and due to electrostatic interactions (Vander Waals forces). Furthermore, the amine groups on amine-derivatized magnetic beads interact with proteins localized on the surface of the bacterial cells in the milk sample. Culturing of magnetic bead suspensions prepared from clinical milk samples on selective agar plates has revealed bacterial colonies, which confirms their binding to bacterial cells.

Regarding the choice of the aqueous solution used to resuspend the beads containing the bound bacterial cells, as noted in the detailed description, the aqueous solution is preferably a buffered aqueous solution compatible with downstream PCR amplification, such as Tris-HCl, MOPS (3-(N-morpholino)propanesulfonic acid), or HEPES (4-(2-hydroxyethyl)-1-piperazineethanesulfonic acid), wherein pH of the buffered aqueous solution is between 8.0 and 9.5. In one desired embodiment, the buffered aqueous solution is stabilized by Tris-HCl which is present at about 20 to 30 mM, and at a pH of around 8 to 9, for example. In the present, non-limiting example, the buffered aqueous solution is 25 mM Tris-HCl, pH 8.5, which is compatible with downstream qPCR. The present inventors also added detergents (Brij-L23, Saponin, CHAPS, SDS), high salts (NaCl), and/or a chelating agent (EDTA) to 25 mM or 50 mM Tris pH 8.5 buffer as indicated below in order to assess if these added components improved performance:
1. 25 mM Tris (pH 8.5)+0.5% Brij-L23
2. 25 mM Tris (pH 8.5)+1.0% Brij-L23
3. 25 mM Tris (pH 8.5)+1.5% Brij-L23
4. 25 mM Tris (pH 8.5)+0.5% Saponin
5. 25 mM Tris (pH 8.5)+1.0% Saponin
6. 25 mM Tris (pH 8.5)+0.5% CHAPS
7. 25 mM Tris (pH 8.5)+50 mM NaCl
8. 25 mM Tris (pH 8.5)+50 mM NaCl+5 mM EDTA+1% SDS
9. 50 mM Tris (pH 8.5)+100 mM NaCl+5 mM EDTA

It was found that the Tris buffered aqueous solutions 1-9 above worked similarly to 25 mM Tris (pH 8.5) without the added detergents, salt, or chelating agent. However, since no significant improvement in the performance was observed, 25 mM Tris buffer (pH 8.5) was selected due to its simplicity.

The Real-time PCR program was also modified by the present inventors to increase the time of the initial denaturation step at 95° C. from 5 minutes to 7.5-10 minutes, during which bacterial cells may be lysed and DNA is released.

Regarding the choice of magnetic beads, it was found that there was no difference in the results obtained when the milk sample preparation method was tested using Dynabeads™ with silica-like surface chemistry, Dynabeads™-streptavidin, lectin-derivatized, or amine-derivatized magnetic beads. Therefore, Dynabeads™ with silica-like surface chemistry were used for subsequent testing due to their simplicity.

Another notable aspect of the milk sample preparation method of this invention is the separation of bacterial cells from clinical milk, which in turn eliminates the inhibitory effects of milk constituents on the downstream real-time PCR assay.

During further optimization, different combinations of milk sample input and magnetic beads were evaluated, and it was observed that a combination of 200 μL of clinical milk samples and 10 μL of magnetic beads appeared promising.

### Example 6—Detection of Pathogens in Milk Samples

A total of 42 archived milk samples collected from mastitic quarters of cows were considered for testing of the rapid milk sample preparation method and a downstream real-time PCR assay as described herein (Table 1). A combination of culture and MALDI-TOF analysis performed on these milk samples identified Gram-positive pathogens such as S. aureus and coagulase-negative staphylococci (CNS). A set of 26 milk samples collected from mastitic quarters of cows with E. coli (Gram-negative) growth were also included to estimate specificity (Table 1). A preliminary assessment of the milk sample preparation method according to the present invention with downstream real-time PCR assays was performed on these 68 clinical milk samples using culture and MALDI-TOF as the reference method.

The Gram-positive specific primers and probes used in the PCR assay are described in Example 4 above. Staphylococcus aureus and coagulase-negative staphylococci (CNS) are Gram-positive bacteria and should be amplified, which provides an indication on sensitivity. On the other hand, E. coli is a Gram-negative bacterium and the Gram-positive specific primers and probes used in the study should not amplify E. coli, which provides an indication on specificity. Culture and MALDI-TOF is the reference method which provides the true status of milk samples for comparison purposes with the methods of this invention.

The outcome of this study indicated that the performance of the bead-free supernatant was superior when compared to the bead suspension as the template (Table 2). Additional molecular assay optimizations and fresh clinical milk samples could improve the assay outcome. Typically, molecular methods detect both dead as well as viable cells. Whereas, the culture method has the potential to demonstrate viable cells only. Therefore, molecular methods appear to have lower specificity, when compared against the culture method.

TABLE 1
Clinical milk samples used in this study

| | | Number of milk |
|---|---|---|
| Type | Etiological agent | samples |
| Gram-positive | Staphylococcus aureus-positive | 19 |
| | CNS-positive | 23 |
| Gram-negative | E. coli-positive | 26 |

TABLE 2
Verification of milk sample preparation method using clinical milk samples

| Sample | True | False | True | False | Sensitivity | Specificity |
|---|---|---|---|---|---|---|
| type | positives | Negatives | Negatives | Positives | (95% CI) | (95% CI) |
| Bead | 25 | 17 | 24 | 2 | 59.5% | 92.3% |
| suspension | | | | | (43.3-74.4%) | (74.9-99.1%) |
| Bead-free | 34 | 8 | 22 | 4 | 81.0% | 84.6% |
| supernatant | | | | | (65.9-91.4%) | (65.1-95.6%) |

In this invention, a rapid milk sample preparation method compatible with downstream real-time PCR assay was successfully developed for the detection of bacterial pathogens in milk. Important advantages relative to the prior art include the following features:
1. This rapid method is feasible for point-of-care applications to detect bacterial pathogens in milk
2. No instrumentation (centrifuge and heating block) is required
3. This method has the potential to reduce inhibitory effects of milk constituents on downstream molecular assay
4. Multiple washing or pipetting steps are not required
5. No requirement of special storage (refrigerated or −20° C.) of buffer
6. Doesn't require use of any hazardous chemical

1. A method of preparing a milk sample for a downstream nucleic acid amplification process, the method comprising:
a) subjecting a milk sample comprising at least one suspected bacterial pathogen implicated in mastitis to contact with uncoated magnetic beads;
b) incubating the milk sample in the presence of the uncoated magnetic beads to allow bacterial cells in the milk sample to bind to the beads, thereby forming a bead-bacterium complex;
c) subjecting the bead-bacterium complex to a magnet such that the bead-bacterium complex is separated from a milk sample supernatant;
d) removing the milk sample supernatant;
e) directly resuspending the separated bead-bacterium complex in an aqueous solution to release the bead-bound bacteria into the solution; and
f) employing an aliquot of the bead resuspension from e) as a template for primer-specific downstream nucleic acid amplification.

2. The method of claim 1, further comprising subjecting the bead resuspension from f) to a magnet to separate the beads from a supernatant, wherein an aliquot of the bead-free supernatant is used as the template for the primer-specific downstream nucleic acid amplification.

3. The method of claim 1, wherein the milk sample is a raw milk sample.

4. The method of claim 3, wherein the raw milk sample is from a mastitic quarter of a dairy cow.

5. The method of claim 1, wherein the bead-bacterium complex in e) is resuspended in a buffered aqueous solution that provides a suitable chemical environment for activity of DNA polymerase.

6. The method of claim 5, wherein pH of the buffered aqueous solution is between 8.0 and 9.5.

7. The method of claim 6, wherein the buffered aqueous solution is stabilized by Tris-HCl, Tris-H₃PO₄, MOPS (3-(N-morpholino)propanesulfonic acid), HEPES (4-(2-hydroxyethyl)-1-piperazineethanesulfonic acid), or combinations thereof.

8. The method of claim 7, wherein the buffered aqueous solution is stabilized by Tris-HCl or Tris-H₃PO₄ which is present at about 10 to about 50 mM and at a pH of about 8.0 to about 9.0.

9. The method of claim 8, wherein the buffered aqueous solution is 25 mM Tris-HCl, pH 8.5.

10. The method of claim 5, wherein the buffered aqueous solution further comprises a chelating agent, a salt, a detergent, or combinations thereof.

11. The method of claim 10, wherein the chelating agent is ethylenediaminetetraacetic Acid (EDTA), ethylene bis(oxyethylenenitrilo)tetraacetic acid (EGTA), or sodium 4-aminosalicylate.

12. The method of claim 10, wherein the detergent is tricosaethylene glycol dodecyl ether (Brij L23), octoxynol-1 (Triton X-100), polyoxyethylene sorbitan monolaurate (Tween-20), triethanolamine (TEA), 3-((3-cholamidopropyl) dimethylammonio)-1-propanesulfonate (CHAPS), Sodium Dodecyl Sulfate (SDS), or combinations thereof.

13. The method of claim 10, wherein the salt is selected from sodium chloride (NaCl), magnesium chloride (MgCl₂), potassium chloride (KCl), or combinations thereof.

14. The method of claim 1, wherein the milk sample and the uncoated magnetic beads are mixed prior to the incubation.

15. The method of claim 1, wherein the incubation is for about 5 to about 10 minutes at room temperature.

16. The method of claim 1, wherein the steps of preparing the milk sample are carried out in a tube.

17. The method of claim 16, wherein the tube is placed in a magnetic stand to capture the magnetic beads.

18. The method of claim 1, wherein the uncoated magnetic beads comprise silica-like surface chemistry.

19. The method of claim 1, wherein the uncoated magnetic beads are surface derivatized with lecithin, streptavidin, or an amine.

20. The method of claim 1, wherein the template is analyzed by real-time PCR using primers specific to a bacterial pathogen implicated in mastitis.

21. The method of any one of claim 20, wherein the bacterial pathogen implicated in mastitis is selected from the group consisting of Staphylococcus aureus, Streptococcus sp. and coagulase-negative staphylococci (CNS).

22. A point-of-care (PoC) molecular test that allows identification of an etiological pathogen in a milk sample, the molecular test comprising the use of a template for primer-specific nucleic acid amplification that has been prepared according to the method of claim 1.

23. The method of claim 22, wherein the milk sample is from a mastitic quarter of a dairy cow.

24. The method of claim 22, wherein the molecular test is real-time PCR.
